(19) United States
(12) Patent Application Publication
(10) Pub. No.: US 2004/0079908 A1
(43) Pub. Date: Apr. 29, 2004

(54) Radiation detecting cassette

(75) Inventor: Takeshi Ohkubo, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/684,705

(22) Filed: Oct. 15, 2003

(30) Foreign Application Priority Data
Oct. 15, 2002 (JP) 300417/2002

(51) Int. Cl. G21K 4/00; A61B 6/00; G01N 23/04

(52) U.S. Cl. 250/582

(57) ABSTRACT

A radiation detecting cassette having a cassette main body, which houses a solid state radiation detector and a control means for controlling the operations of the solid state radiation detector, and an operating portion for inputting commands for operating the solid state radiation detector is provided. The radiation detecting cassette is easily set in a radiation image obtaining apparatus, enables easy operations using the operating portion, and enables operations using the operating portion at positions remote from the radiation image obtaining apparatus. The operating portion is formed as a separate unit from the cassette main body. A display portion provided at the operating portion displays operating states of the solid state radiation detector, image processing conditions, images based on image signals output from the solid state radiation detector, and the like.

### BACKGROUND OF THE INVENTION

1. Field of the Invention

The present invention relates to a radiation detecting cassette comprising: a solid state radiation detector for detecting radiation and outputting an image signal representing a radiation image; and a case for housing the solid state radiation detector.

2. Description of the Related Art

Presently, various solid state radiation detectors, used in radiation imaging for medical diagnosis and the like, have been proposed and are in practical use (refer to, for example, U.S. Pat. No. 6,268,614). The solid state radiation detectors detect radiation transmitted through a subject, then output image signals representing radiation images of the subject.

Radiation detecting cassettes, comprising a solid state radiation detector; an image memory for recording the image signals output by the solid state radiation detector; and image processing means for administering image processes on the image signals, have also been proposed (refer to, for example, U.S. Pat. No. 6,344,652).

The radiation detecting cassette disclosed in U.S. Pat. No. 6,344,652 further comprises an operating portion for selecting and displaying image processing conditions. In this radiation detecting cassette, the operating portion is provided on a surface opposite from the radiation detecting surface.

In the radiation detecting cassette provided with the operating portion described above, the operating portion is provided on the cassette itself. In order to maintain operability, the operating portion is required to be of at least a certain size. Therefore, in the case that a cassette insertion portion of a radiation image obtaining apparatus is not compatible with the radiation detecting cassette described above, it may be physically impossible to set the radiation detecting cassette in the apparatus, due to the operating portion getting in the way. In addition, even if the radiation detecting cassette is set in the cassette insertion portion of the radiation image obtaining apparatus, operation of the operating portion becomes difficult. This is due to the operating portion being positioned beneath an imaging table in the case of prone imaging, or the operating portion being positioned behind the imaging table in the case of standing imaging. In addition, if portable imaging is performed, in which case the radiation detecting cassette is set between the subject and a subject placement table (e.g., an imaging table, a bed), the operating portion is positioned between the subject and the subject placement table. Therefore, operations, such as changing of image processing conditions using the operating portion, cannot be performed. Further, operations cannot be performed using the operating portion at a position remote from the radiation image obtaining apparatus, such as outside an imaging room.

### SUMMARY OF THE INVENTION

The present invention has been developed in view of the points described above. It is an object of the present invention to provide a radiation detecting cassette provided with an operating portion, which is easily set in a radiation image obtaining apparatus, which enables operations to be performed using the operating portion easily, and which enables operations to be performed using the operation portion at positions remote from the radiation image obtaining apparatus.

The radiation detecting cassette of the present invention comprises:

a solid state radiation detector for detecting radiation bearing image information and outputting an image signal representing a radiation image;

a control means for controlling the operations of the solid state radiation detector;

a cassette main body having a case for housing the solid state radiation detector and the control means; and

a portable operating portion for outputting command signals to the control means for operating the solid state radiation detector, formed as a separate unit from the cassette main body.

Here, the “solid state radiation detector” may be of any structure so long as it detects radiation bearing image information and outputs an image signal representing a radiation image. For example, there are solid state radiation detectors that directly convert radiation incident thereon to electric charges, and those that convert radiation incident thereon to light, then convert the light to electric charges. An image signal representing a radiation image is obtained by outputting the charges. In addition, the “solid state radiation detector” includes those which comprise a solid state radiation detector main body and image processing means for administering image processes on an output image signal.

The “operations of the solid state radiation detector” include the operations of the solid state radiation detector during recording of a radiation image; during readout of a radiation image; during administration of image processes on an image signal output from the radiation solid state detector main body; and the like.

The “command signals . . . for operating the solid state radiation detector” include commands for readying the solid state detector to record a radiation image; readying the solid state detector to read out a radiation image therefrom; administering image processes on an image signal output from the solid state radiation detector main body; changing the contents of image processes; changing the image processing conditions; and the like. Examples of image processes include: a gradation process; a frequency emphasis process; a density/contrast standardizing process; a noise suppressing process; a grid pattern removing process; a blackening process for areas outside of an irradiated field; an energy subtraction process, and a time-lapse subtraction process. The “contents of image processes” refers to the administration of an image process or image processes from among various image processes such as those listed above. The “image processing conditions” refer to settings (such as image processing parameters) of each image process to be administered.

“Portable” means that an operator can use the operating portion in a hand held manner. The operating portion of the radiation solid state detector according to the present invention does not include those which are mounted at predetermined positions, or those which are wheeled, and moved by being pushed.

The operating portion may further comprise a display portion for displaying the command signals.

The operating portion may further comprise an information receiving means for receiving information output from the cassette main body. In this case, the display portion may display the information received by the information receiving means.

Here, the “information output from the cassette main body” may be any information output by the cassette main body. For example, the information may be that which represents an operating state of the solid state radiation detector (e.g., recording of a radiation image, readout of a radiation image, administering of image processes); information received by the cassette main body from an imaging apparatus (e.g., patient information and test results); and the like.

The information receiving means may receive image signals output from the solid state radiation detector. In this case, the display portion may display images based on the image signals output from the solid state radiation detector.

Here “images” include reduced images, to which reduction processes have been administered, images based on image signals to which predetermined image processes have been administered, and the like.

The operation portion may be removably attachable to the case.

According to the radiation detecting cassette of the present invention, the portable operating portion is formed as a separate unit from the cassette main body. Therefore, only the cassette main body may be set into the cassette insertion portion of a radiation image obtaining apparatus, without the operating portion getting in the way. Operations using the operating portion are easily performed even in the case that the radiation detecting cassette is set in a radiation image obtaining apparatus for prone imaging, because only the cassette main body is set therein. In addition, operations using the operating portion are enabled at a position remote from the radiation image obtaining apparatus, because only the cassette main body is set therein.

In the case that the commands for operating the solid state radiation detector are displayed at the display portion of the operating portion, the commands can be visually confirmed while being input.

In the case that information output from the cassette main body is displayed at the display portion of the operating portion, an operating state of the solid state radiation detector, for example, can be known. Therefore, operations can be performed efficiently and accurately.

In the case that images, based on image signals output from the solid state radiation detector, are displayed at the display portion of the operating portion, contents of image processes, image processing conditions, and the like can be changed while viewing the displayed images. In addition, faulty imaging can be confirmed immediately, at the imaging location.

In the case that the operating portion is removably attachable to the case, portability and convenience in storage are improved by integrating the operating portion with the cassette main body.

### BRIEF DESCRIPTION OF THE DRAWINGS

FIG. 1 is a schematic view showing the construction of a radiation detecting cassette according to an embodiment of the present invention.

FIG. 2 is a plan view of a cassette main body of the radiation detecting cassette of FIG. 1.

FIG. 3 is a schematic view showing the construction of a radiation image obtaining apparatus employing the radiation detecting cassette of FIG. 1.

### DESCRIPTION OF THE PREFERRED EMBODIMENTS

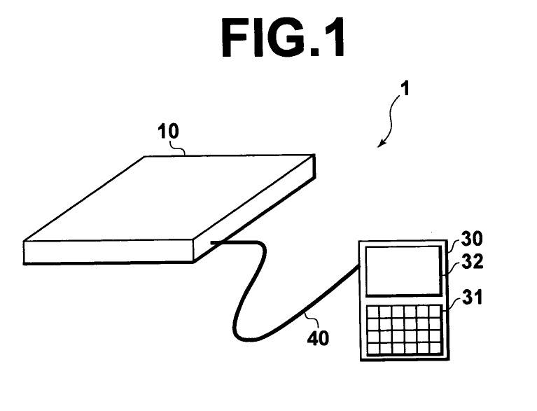

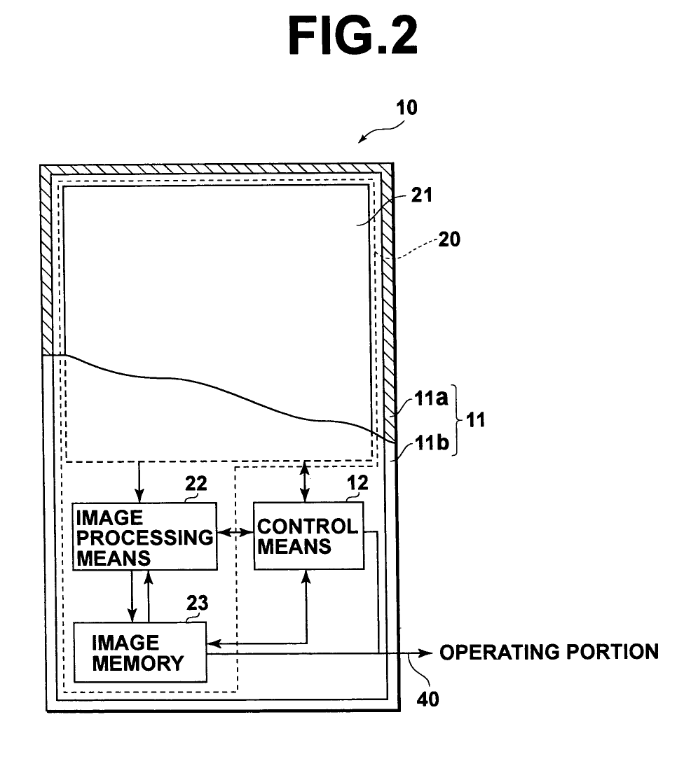

Hereinafter, an embodiment of the present invention will be described with reference to the attached drawings. FIG. 1 is a schematic view showing the construction of a radiation detecting cassette according to an embodiment of the present invention. FIG. 2 is a plan view of a cassette main body 10 of the radiation detecting cassette, shown with a lid portion partially omitted.

As shown in FIG. 1, the radiation detecting cassette 1 comprises: the cassette main body 10; and an operating portion 30 for outputting command signals for operating a solid state radiation detector 20, formed as a separate unit from the cassette main body 10. The cassette main body 10 and the operating portion 30 are connected by a cable 40.

As shown in FIG. 2, the cassette main body 10 comprises: the solid state radiation detector 20 for detecting radiation bearing image information and outputting an image signal representing a radiation image; a control means 12 for controlling the operations of the solid state radiation detector 20; and a substantially parallelepiped case 11 for housing the solid state radiation detector 20 and the control means 12.

As shown in FIG. 2, the solid state radiation detector 20 comprises: a solid state radiation detector main body 21; an image processing means 22, for administering predetermined image processes to image data output from the solid state radiation detector main body 21, connected to thereto; and an image memory 23 for recording the image signal output from the solid state radiation detector main body 21, or the image signal which has been processed by the image processing means 22.

Note that in FIG. 2, the solid state radiation detector main body 21, the image processing means 22, the image memory 23, and the control means 12 are arranged to facilitate understanding of the structure of the solid state radiation detector 20. In actuality, the image processing means 22, the image memory 23, the control means 12, and the like are placed behind the solid state radiation detector main body 21 (the side opposite the surface on which radiation is incident), to miniaturize the radiation detecting cassette main body 10.

The solid state radiation detector main body 21 may be of any construction as long as it detects radiation bearing image information of a subject, and outputs an image signal representing a radiation image of the subject. An example of such a solid state radiation detector main body comprises: a first electrode layer that transmits recording radiation; a recording light photoconductive layer that exhibits conductivity when irradiated with radiation; a charge transport layer that acts substantially as an insulator with respect to latent image charges, and acts substantially as a conductor with respect to transport charges of a polarity opposite that of the latent image charges; a reading light photoconductive layer that exhibits conductivity when irradiated with reading electromagnetic waves; and a second electrode layer that transmits the reading electromagnetic waves; layered in the order listed. The solid state radiation detector main body described above records radiation image information in the following manner. Recording radiation is irradiated from the side of the first electrode layer, and charges corresponding to the amount of radiation are accumulated at an accumulation portion formed at the interface between the recording light photoconductive layer and the charge transport layer. The radiation image information recorded in manner described above is read out by scanning the solid state radiation detector main body with spot light or linear light. A mechanism for scanning the readout light (not shown) is also provided in the case 11. As an alternative to the direct conversion type solid state radiation detector main body described above, an indirect conversion type solid state radiation detector main body may be utilized. The indirect conversion type solid state radiation detector main body records radiation image information in the following manner. Phosphorescence, emitted by stimulable phosphors by irradiation of radiation thereon, is irradiated on a recording light photoconductive layer. The phosphorescence is photoelectrically converted to obtain signal charges, which are accumulated at an accumulating portion. As a further alternative, a solid state radiation detector main body which reads out charges, generated at a light photoconductive layer by irradiation of radiation thereon, by scanning thin film transistors (TFT) may be employed.

The case 11 comprises: a boxlike housing portion 11a which has an open upper surface; and a lid 11b for covering the upper surface of the housing portion 11a.

The operating portion 30 comprises: an operating panel 31 for inputting commands for operating the solid state radiation detector 20; and a display portion 32 for displaying an operating state of the solid state radiation detector 20, reduced images based on image signals output from the solid state radiation detector 20, and the like. The operating panel is a keyboard comprising alphanumeric keys, cursor keys, and the like. The display portion 32 comprises an LCD panel.

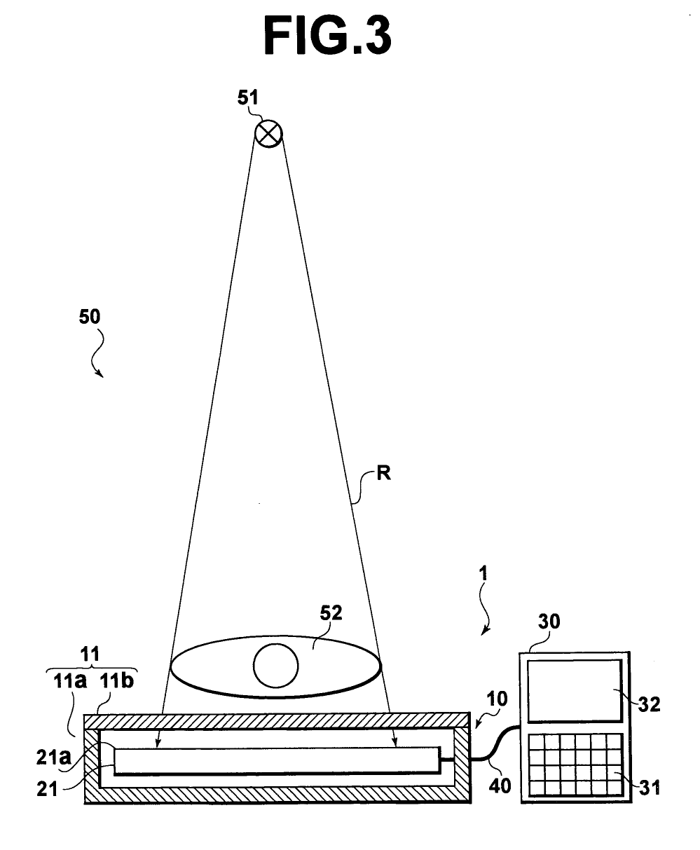

Next, the operation of the radiation detecting cassette 1 of the present invention will be described, using radiation imaging of the chest as an example. FIG. 3 is a schematic view showing the construction of a radiation image obtaining apparatus 50 employing the radiation detecting cassette 1, along with the subject of imaging.

The radiation image obtaining apparatus 50 comprises a radiation source 51, and is configured to enable the radiation detecting cassette 1 to be set therein. The cassette main body 10 is placed so that radiation, which is emitted from the radiation source 51 and passes through the subject 52, is incident on a detecting surface 21a of the solid state radiation detector main body 21.

First, a command signal to switch to an image recording mode is output from the operating portion 30. The command signal is input to the control means 12 of the cassette main body 10. The control means 12 switches the solid state radiation detector 20 to the image recording mode, according to the input command signal. Switching to the image recording mode refers to readying the solid state radiation detector 20 so that it is capable of recording a radiation image. For example, the switching involves applying a voltage between the first electrode layer and the second electrode layer of the solid state radiation detector main body 21. At this time, a message or the like, indicating that a command to switch to the image recording mode has been issued, is displayed at the display portion 32.

Next, an imaging menu is input to an imaging control apparatus (not shown). The imaging menu is input to the control means 12 of the cassette main body 10. Pre-registered image processing conditions and the like are set in the image processing means 22, according to the imaging menu. Then, the imaging menu is output from the cassette main body 10 to an information receiving means (not shown) of the operating portion 30. The imaging menu received by the information receiving means is displayed at the display portion 32. In the case that an operator wishes to change the registered image process or the like, settings are changed by operating the operating panel 31. Note that the imaging menu refers to at least one of an imaging portion (head, chest, upper limb, lower limb, etc.), an imaging method (simple imaging, tomographic imaging, contrast imaging, enlargement imaging, etc.), or a combination thereof. The display portion 32 of the operating portion 30 may display test results, patient information, and the like, received by the information receiving means from the imaging control apparatus, in addition to the imaging menu.

Radiation is emitted from the radiation source 51, according to a control signal from the imaging control apparatus, and imaging is performed according to imaging conditions set in the imaging menu. Then, an image signal, based on a radiation image recorded by the solid state radiation detector 20, is output to the image memory 23 and recorded therein. During imaging, information representing the operating state of the solid state radiation detector 20 is output from the control means 12 of the cassette main body 10 to the operating portion 30. The information representing the operating state is received by the information receiving means of the operating portion 30, and messages, indicating the operating states of radiation image recording and output of the image signal, are displayed at the display portion 32. The image signal recorded in the image memory 23 is output to the image processing means 22, and image processes are administered to the image signal by the image processing means 22. The processed image signal is recorded with the original image signal in the image memory 23. During the image processes, information indicating that image processes are being administered is output from the control means 12 to the operating portion 30. The information indicating that image processes are being administered is received by the information receiving means of the operating portion 30, and a message, indicating that image processes are being administered, is displayed at the display portion 32.

Next, a reduction process is administered to the processed image signal recorded in the image memory 23, and a reduced image signal is output to the information receiving means of the operating portion 30. The display portion 32 displays a reduced image, based on the reduced image signal received by the information receiving means.

The operator views the reduced image displayed on the display portion 32 and determines whether to change the image processing conditions or the like. In the case that the operator determines that changing the image processing conditions or the like is necessary, new image processing conditions are input via the operating panel 31. The new image processing conditions are output to the image processing means 22. The original image signal is output from the image memory 23 to the image processing means 22. After image processes are administered again, the reduction process is administered, and a reduced image, to which image processes have been administered with the new image processing conditions, is displayed at the display portion 32 of the operating portion 30.

In the case that a desired reduced image is displayed at the display portion 32, a list of apparatuses which are capable of receiving the image signal is displayed at the display portion 32. A destination apparatus for the image signal, an order of transmission, and the like are specified via the operating panel 31. A diagnostic image display terminal, a film output apparatus, and an image data filing apparatus are examples of the destination apparatus.

A command signal to transmit the image signal is input via the operating panel 31. The image signal transmit command is input to the cassette main body 10. The control means 12 outputs the processed image signal from the image memory 23 to an external apparatus, such as a diagnostic image display terminal, a film output apparatus, and an image data filing apparatus. Then, the radiation image based on the processed image signal is displayed on a monitor of the diagnostic image display terminal, the radiation image based on the processed image signal is recorded on film by the film output apparatus, the processed image signal is recorded in a server by the image data filing apparatus, and the like. Information representing that the processed image signal is being output is output from the control means 12 to the operating portion 30 and received by the information receiving means of the operating portion 30. A message, indicating that the image signal is being transmitted, is displayed at the display portion 32.

According to the radiation detecting cassette 1 described above, the operating portion 30 is formed as a separate unit from the cassette main body 10. Therefore, only the cassette main body 10 may be set into the cassette insertion portion of a radiation image obtaining apparatus, without the operating portion 30 getting in the way. Operations using the operating portion 30 are easily performed even in the case that the radiation detecting cassette 1 is set in a radiation image obtaining apparatus for prone imaging, because only the cassette main body 10 is set therein. In addition, operations using the operating portion 30 are enabled at a position remote from the radiation image obtaining apparatus, because only the cassette main body 10 is set therein.

Because the commands for operating the solid state radiation detector 20 are displayed at the display portion 32 of the operating portion 30, the commands can be visually confirmed while being input.

Because the operating state of the solid state radiation detector 20 is displayed at the display portion 32 of the operating portion 30, operations can be performed efficiently and accurately.

Because the image, based on the image signal output from the solid state radiation detector 20, is displayed at the display portion 32 of the operating portion 30, image processing conditions and the like can be changed while viewing the displayed image.

In the embodiment described above, the cassette main body 10 and the operating portion 30 are connected by the cable 40. Alternatively, wireless connections, utilizing infrared beams or other electromagnetic waves, may be employed. In the case that a wireless connection is employed, channel switching should be enabled, to eliminate crosstalk with other radiation detecting cassettes.

Regarding the operating states of the solid state radiation detector 20, which are displayed at the display portion 32, they are not limited to those described above. Other operating states may be displayed as well.

The display portion 32 in the embodiment described above is a liquid crystal panel. However, the display portion is not limited to this construction, and may be any display device, such as an organic EL display, as long it is capable of forming the operating portion 30 at a portable size.

The display portion 32 may display color images. However, the images obtained from the solid state radiation detector 20 are gray scale images. Therefore, the display pixels assigned to each color of RGB may be caused to emit light in a gray scale, to display gray scale images at high resolution.

ID data of the cassette main body 10 may be transmitted therefrom to be received by the operating portion 30, or ID data stored within the operating portion 30 may be read out and displayed on the display portion 32. Thereby, the correspondence between operating portions and cassette main bodies may be understood, in the case that a plurality of each exists.

A configuration may be adopted wherein the control means 12 of the cassette main body 10 does not respond to commands from the operating portion 30 during recording or readout of a radiation image onto or from the solid state radiation detector. Alternatively, a configuration may be adopted wherein the operating portion 30 is precluded from outputting command signals during a period of time from reception of data indicating the performance of a recording operation or a readout operation to data indicating that the operation is complete. In these cases, noise due to operations being performed other than recording or readout of the radiation image during recording or readout thereof, being included in the image signal, can be prevented.

The operating portion 30 may be removably attachable to the cassette main body 10. In this case, any structure may be adopted, as long as the operating portion 30 is removably attachable to the cassette main body 10. In addition, in the case that the operating portion 30 and the cassette main body 10 are connected via a wireless connection, a configuration may be adopted in which the operating portion 30 is charged by the cassette main body 10 when they are integrated. If this configuration is adopted, the necessity for a power source for the operating portion 30 is obviated.

What is claimed is:

1. A radiation detecting cassette comprising:
a solid state radiation detector for detecting radiation bearing image information and outputting an image signal representing a radiation image;
a control means for controlling the operations of the solid state radiation detector;
a cassette main body having a case for housing the solid state radiation detector and the control means; and
a portable operating portion for outputting command signals to the control means for operating the solid state radiation detector, formed as a separate unit from the cassette main body.

2. A radiation detecting cassette as defined in claim 1, wherein:
the operating portion further comprises a display portion for displaying the contents of the command signals.

3. A radiation detecting cassette as defined in claim 2, wherein:
the operating portion further comprises an information receiving means for receiving information output from the cassette main body; and
the display portion displays the information received by the information receiving means.

4. A radiation detecting cassette as defined in claim 3, wherein:
the information receiving means receives information representing an operating state of the solid state radiation detector; and
the display portion displays the operating state of the solid state radiation detector.

5. A radiation detecting cassette as defined in claim 3, wherein:
the information receiving means receives the image signal output from the solid state radiation detector; and
the display portion displays an image based on the image signal.

6. A radiation detecting cassette as defined in claim 4, wherein:
the information receiving means receives the image signal output from the solid state radiation detector; and
the display portion displays an image based on the image signal.

7. A radiation detecting cassette as defined in claim 1, wherein:
the operating portion is removably attachable to the case.

8. A radiation detecting cassette as defined in claim 2, wherein:
the operating portion is removably attachable to the case.

9. A radiation detecting cassette as defined in claim 3, wherein:
the operating portion is removably attachable to the case.

10. A radiation detecting cassette as defined in claim 4, wherein:
the operating portion is removably attachable to the case.

11. A radiation detecting cassette as defined in claim 5, wherein:
the operating portion is removably attachable to the case.

12. A radiation detecting cassette as defined in claim 6, wherein:
the operating portion is removably attachable to the case.
